(19) United States
(12) Patent Application Publication
(10) Pub. No.: US 2005/0084524 A1
(43) Pub. Date: Apr. 21, 2005

(54) Method for potentiating activity of a chemotherapeutic drug

(75) Inventors: Francis J. Martin, San Francisco, CA (US); Gail T. Colbern, Pacifica, CA (US); Peter K. Working, Burlingame, CA (US)

Correspondence Address: PERKINS COIE LLP, P.O. BOX 2168, MENLO PARK, CA, 94026, US

(73) Assignee: ALZA Corporation

(21) Appl. No.: 10/964,059

(22) Filed: Oct. 12, 2004

Related U.S. Application Data
(63) Continuation of application No. 09/408,080, filed on Sep. 29, 1999, now abandoned.
(60) Provisional application No. 60/102,489, filed on Sep. 30, 1998.

(51) Int. Cl. A61K 39/395; A61K 33/24; A61K 9/127; A61K 31/704

(52) U.S. Cl. 424/450; 424/649; 514/34; 424/143.1

(57) ABSTRACT

A method for potentiating the activity of a chemotherapeutic drug administered in combination with a biological agent is described. The method includes entrapping the chemotherapeutic drug in a liposome and administering the liposome-entrapped drug in combination with the biological agent. The method is particularly useful for treatment of cancer which over-express tyrosine kinase receptor and for B-cell lymphomas, where, for example, anti-HER2 antibodies or anti-CD19 antibodies are administered in combination with the cytotoxic drug.

This application claims the priority of provisional application Ser. No. 60/102,489, filed Sep. 30, 1998, and which is incorporated herein by reference.

### FIELD OF THE INVENTION

The present invention relates to a method of potentiating the activity of a liposome-entrapped anticancer compound by administering a biological agent in conjunction with the liposome-entrapped anticancer compound.

### BACKGROUND OF THE INVENTION

It is estimated that one-third of all individuals in the United States will develop cancer. The 5-year relative survival rate for these individuals, that is the probability of escaping death from cancer for 5 years following diagnosis, has risen to nearly 50% as a result of progress in the early diagnosis and the therapy of the disease. However, cancer remains second only to cardiac disease as a cause of death in this country. The three most common types of cancer are cancers of the lung, breast and colo-rectal (Harrison's Principles of Internal Medicine, 12^{th} Edition, McGraw-Hill, Inc., 1991).

Modern technology has armed the medical world with an array of anticancer compounds and, more recently, biological agents which act at a cellular level through specific interactions with the genes, the proteins encoded by the genes or the cell surface receptors. For example, the HER2 gene (also known as neu and as c-erbB-2) encodes a 185 kDa transmembrane tyrosine/kinase receptor designated p185^{HER2}. HER2 is overexpressed in 25-30% of breast, lung and ovarian cancers. Antibodies directed at p185^{HER2}, such as the recombinant humanized anti-p185^{HER2} antibody Herceptin® which binds to the extracellular domain of the receptor, can inhibit the growth of tumors and of transformed cells that express high levels of the p185^{HER2} receptor (Hudziak, et al.; U.S. Pat. No. 5,772,997; Baselga, J. et al., Cancer Research, 58:2825 (1998); Baselga, J. et al., J. Clinical Oncology, 14(3):737 (1996)).

Another biological agent is a humanized monoclonal antibody directed against epidermal growth factor receptor (EGFR), which is implicated in breast cancer (Chrysogelos, S. A. et al., Breast Cancer Res. and Treatment, 29:29-40 (1994)).

Another biological agent that has been described is an antibody which targets the CD20 antigen in B cells (Anderson, et al, U.S. Pat. No. 5,776,456). B-cell lymphocytes are responsible for antibody production in response to an invading antigen. Occasionally, proliferation of a particular B cell occurs and results in a cancer referred to a B cell lymphoma. CD20 is a cell surface protein expressed during early pre-B-cell development and remaining until plasma cell differentiation. Thus, the CD20 surface antigen has the potential of serving as a candidate for targeting of B-cell lymphomas.

To date, administration of these and other biological agents has not been an entirely effective cancer therapy and coadministration of the biological agent with a more traditional chemotherapeutic drug has been suggested (Hudziak, et al.; U.S. Pat. No. 5,772,997; Chen, et al., U.S. Pat. No. 5,773,476). Traditional chemotherapeutic drugs include vinblastine, actinomycin D, etoposide, cisplatin, methotrexate, doxorubicin, paclitaxel, and 5-fluorouracil. One problem with this approach is in the severity of the resulting toxicity, including an increase in the frequency and severity of nausea, vomiting, neutropenia, mucositis, alopecia, and cardiotoxicity. This is especially a problem with frail patient populations, such as children and the elderly. There is still, then, a need for an improved cancer therapy.

### SUMMARY OF THE INVENTION

Accordingly, it is an object of the invention to enhance the therapeutic activity of an anticancer compound.

It is another object of the invention to enhance the activity of an anticancer compound with no increase in toxicity.

Yet another object of the invention is to improve the chemo-sensitization activity of a chemotherapeutic drug, and more specifically to enhance the tumor cell chemo-sensitization to the drug.

Still another object of the invention is to provide a method for potentiating the antitumor activity of a therapeutic compound when a subtherapeutic amount of the compound is administered.

In one aspect, the invention includes a method for potentiating the activity of a chemotherapeutic drug administered in combination with a biological agent to a subject suffering from cancer. The method includes entrapping the chemotherapeutic drug in a liposome, and administering the liposome-entrapped drug in combination with the biological agent.

In one embodiment of the method, the liposome-entrapped drug is an anthracycline antibiotic, such as doxorubicin, daunorubicin, epirubicin, idarubicin and analogs thereof. In another embodiment, the liposome-entrapped drug is a platinum-containing compound, such as cisplatin, carboplatin and other derivatives of cisplatin.

The method of the invention, in one embodiment, is for treating a cancer characterized by over-activity of a tyrosine kinase receptor and where the biological agent is capable of binding to such a receptor. For example, the tyrosine kinase receptor can be HER2, epidermal growth factor (EGF) or platelet-derived growth factor (PDGF). Biological agents for such binding include anti-HER2 antibody, anti-EGFR antibody and anti-PDGFR antibody.

In another embodiment, the method of the invention is for use in treating a cancer derived from a B-cell malignancy. The biological agent administered in combination with the liposome-entrapped chemotherapeutic agent is capable of binding to a B-cell surface antigen such as CD19, CD20, CD22 or CD77. More specifically, the biological agent is anti-CD19 antibodies, anti-CD20 antibodies, anti-CD22 antibodies or anti-CD77 antibodies.

In another embodiment, the biological agent is an anti-angiogenesis agent, such as angiostatin, endostatin and oncostatin.

The biological agent is administered concurrently with the liposome-entrapped drug, in one embodiment, or is administered after administration of the liposome-entrapped-drug.

The liposomes, in one embodiment, include a surface coating of hydrophilic polymer chains effective to extend the blood circulation lifetime of the liposomes. In this embodiment, the biological agent is preferably administered after administration of the liposome-entrapped drug.

A preferred liposome composition for use in the method of the invention is composed of liposome-entrapped doxorubicin, where the liposomes have a surface coating of polyethyleneglycol polymer chains. This composition can be coadministered, for example, with anti-HER2 antibody for treatment of cancer cells expressing the HER2 receptor or with an anti-CD20 antibody for treatment of a B-cell lymphoma.

Another preferred liposome composition for use in the method of the invention is composed of liposome-entrapped cisplatin, where the liposomes have a surface coating of polyethyleneglycol polymer chains. This composition can be coadministered, for example, with an anti-HER2 antibody for treatment of cancer cells expressing the HER2 receptor.

In another aspect, the invention includes a method of treating a subject for a cancer derived from over-expression of a tyrosine kinase receptor, by administering to the subject (i) a sub-therapeutic amount of an anthracycline antibiotic entrapped in liposomes formed of a vesicle-forming lipid and including a lipid derivatized with a hydrophilic polymer chain to form a liposome-surface coating of hydrophilic polymer chains, and (ii) a dose of a biological agent having binding activity with tyrosine-kinase receptors on the cancer cells, where the dose of biological agent is effective to potentiate the antitumor activity of the liposome-entrapped antibiotic. In another aspect, the invention includes a method of treating a subject having a B-cell-derived lymphoma, by administering to the subject (i) a sub-therapeutic amount of an anthracycline antibiotic entrapped in liposomes formed of a vesicle-forming lipid and including a lipid derivatized with a hydrophilic polymer chain to form a liposome-surface coating of hydrophilic polymer chains, and (ii) a dose of a biological agent having binding activity to surface epitopes on cells of the B-cell derived lymphoma, where the dose of biological agent being is effective to potentiate the antitumor activity of the liposome-entrapped antibiotic.

In another aspect, the invention includes a method of treating a subject suffering from cancer by administering to the subject a chemotherapeutic agent entrapped in a liposome; and administering an antiangiogenesis biological agent.

These and other objects and features of the invention will be more fully appreciated when the following detailed description of the invention is read in conjunction with the accompanying drawings.

### BRIEF DESCRIPTION OF THE DRAWINGS

FIG. 1 is a plot showing the tumor volume, in mm³, in mice as a function of time following tumor implantation, in days, in response to treatment with saline (closed squares), free doxorubicin (open circles), liposome-entrapped doxorubicin (closed diamonds), anti-EGFR antibody C225 (open squares), anti-EGFR C225 antibody+free doxorubicin (closed circles) and anti-EGFR C225 antibody+liposome-entrapped doxorubicin (open pentagons);

FIG. 2A is a plot showing tumor volume of BT474 xenografts, in mm³, in mice as a function of time following tumor inoculation, in days, in response to treatment with saline (closed squares), free doxorubicin at 4 mg/kg (closed triangles), liposome-entrapped doxorubicin at 3 mg/kg (inverted triangles), anti-HER2 antibody at 10 mg/kg (closed diamonds), anti-HER2 antibody+free doxorubicin (closed circles) and anti-HER2 antibody+liposome-entrapped doxorubicin (open squares);

FIG. 2B is a plot of the data presented in FIG. 2A, where the y-axis scale is from 0-100 mm³ to better visualize the combination treatments;

FIG. 3A is a plot showing tumor volume of BT474 xenografts, in mm³, in mice as a function of time following tumor inoculation, in days, in response to treatment with saline (closed squares), free doxorubicin at 5 mg/kg (closed triangles), liposome-entrapped doxorubicin at 5 mg/kg (inverted triangles), anti-HER2 antibody at 3 mg/kg (closed diamonds), anti-HER2 antibody+free doxorubicin (closed circles) and anti-HER2 antibody+liposome-entrapped doxorubicin (open squares);

FIG. 3B is a plot of the data presented in FIG. 3A, where the y-axis scale is from 0-150 mm³ to better visualize the combination treatments;

FIG. 4A is a plot showing tumor volume of BT474 xenografts, in mm³, in mice as a function of time following tumor inoculation, in days, in response to treatment with saline (closed squares), free doxorubicin at 3 mg/kg (closed triangles), liposome-entrapped doxorubicin at 3 mg/kg (inverted triangles), anti-HER2 antibody at 1 mg/kg (closed diamonds), anti-HER2 antibody+free doxorubicin (closed circles) and anti-HER2 antibody+liposome-entrapped doxorubicin (open squares);

FIG. 4B is a plot of the data presented in FIG. 4A, where the y-axis scale is from 0-50 mm³ to better visualize the combination treatments;

FIG. 5A is a plot showing tumor volume of MDA453 xenografts, in mm³, in mice as a function of time following tumor inoculation, in days, in response to treatment with saline (closed squares), free doxorubicin at 5 mg/kg (closed triangles), liposome-entrapped doxorubicin at 5 mg/kg (inverted triangles), anti-HER2 antibody at 5 mg/kg (closed diamonds), anti-HER2 antibody+free doxorubicin (closed circles) and anti-HER2 antibody+liposome-entrapped doxorubicin (open squares);

FIG. 5B is a plot of the data presented in FIG. 5A, where the y-axis scale is from 0-75 mm³ to better visualize the combination treatments;

FIG. 6A is a plot showing tumor volume of B585 xenografts, in mm³, in mice as a function of time following tumor inoculation, in days, in response to treatment with saline (closed squares), free doxorubicin at 4 mg/kg (closed triangles), liposome-entrapped doxorubicin at 4 mg/kg (inverted triangles), anti-HER2 antibody at 3 mg/kg (closed diamonds), anti-HER2 antibody+free doxorubicin (closed circles) and anti-HER2 antibody+liposome-entrapped doxorubicin (open squares);

FIG. 6B is a plot of the data presented in FIG. 6A, where the y-axis scale is from 0-750 mm³ to better visualize the combination treatments;

FIG. 7A is a plot showing tumor volume of BT474 xenografts, in mm³, in mice as a function of time following tumor inoculation, in days, in response to treatment with saline (closed squares), free cisplatin at 6 mg/kg (closed triangles), liposome-entrapped cisplatin at 6 mg/kg (inverted triangles), anti-HER2 antibody at 3 mg/kg (closed diamonds), anti-HER2 antibody+free cisplatin (closed circles) and anti-HER2 antibody+liposome-entrapped cisplatin (open squares);

FIG. 7B is a plot of the data presented in FIG. 7A, where the y-axis scale is from 0-100 mm³ to better visualize the combination drug+antibody treatments;

FIG. 8 is a plot showing tumor volume of BT474 xenografts, in mm³, in mice as a function of time following tumor inoculation, in days, in response to treatment with saline (closed squares), free cisplatin at 4 mg/kg (closed triangles), liposome-entrapped cisplatin at 4 mg/kg (inverted triangles), anti-HER2 antibody at 0.5 mg/kg (closed diamonds), anti-HER2 antibody+free cisplatin (closed circles) and anti-HER2 antibody+liposome-entrapped cisplatin (open squares);

FIG. 9A is a plot showing tumor volume of BT474 xenografts, in mm³, in mice as a function of time following tumor inoculation, in days, in response to treatment with saline (closed squares), free cisplatin at 4 mg/kg (closed triangles), liposome-entrapped cisplatin at 4 mg/kg (inverted triangles), anti-HER2 antibody at 1 mg/kg (closed diamonds), anti-HER2 antibody+free cisplatin (closed circles) and anti-HER2 antibody+liposome-entrapped cisplatin (open squares); and

FIG. 9B is a plot of the data presented in FIG. 9A, where the y-axis scale is from 0-30 mm³ to better visualize the combination drug+antibody treatments.

### DETAILED DESCRIPTION OF THE INVENTION

I. Definitions

Unless otherwise indicated, the terms below have the following meaning:

“Biological agent” refers to a therapeutic agent of biological origin, vis a vis chemically synthesized drugs. The biological agent generally produces low toxicity when administered systemically, and when given to cancer patients a biological agent is not directly cytotoxic to tumor cells (or the neovascular endothelial cells which provide the blood supply to tumors), but does inhibit or slow their growth after interaction with specific molecular targets expressed on or in the cells. The agent produces cytostasis rather than cytotoxicity; i.e., the agent produces a biological response which is generally not sufficient to provide a meaningful clinical benefit.

“Administering in combination with” or “coadministering” refers to administration of two agents, e.g., typically a cytotoxic or chemotherapeutic drug and a biological agent. The terms include the two compounds being administered by the same or different routes simultaneously or sequentially at any selected time interval, e.g., minutes or hours or days apart.

“Sub-therapeutic amount” refers to a dosage of a compound less than that which would normally be expected to achieve a therapeutic response.

II. Description of the Method

As mentioned above, in one aspect, the invention includes a method of potentiating tumor cell chemosensitivity to a chemotherapeutic drug administered in combination with a biological agent. As will be described below, it has been surprisingly discovered that entrapping the chemotherapeutic agent in a liposome and administering the liposome-entrapped agent in combination with a biological agent results in a synergistic enhancement of the therapeutic effect.

A. Liposome-Entrapped Anticancer Agent

Liposomes are small, spherical vesicles composed of lipids, and usually composed of vesicle-forming lipids, which refer to lipids capable of spontaneously arranging into lipid bilayer structures in water. The vesicle-forming lipids of this type are preferably ones having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol.

In one embodiment of the invention, the liposomes include as one of the vesicle-forming lipid components a vesicle-forming lipid derivatized with a hydrophilic polymer. As has been described, for example in U.S. Pat. No. 5,013,556 and in WO 98/07409, which are hereby incorporated by reference, such a hydrophilic polymer provides a surface coating of hydrophilic polymer chains on both the inner and outer surfaces of the liposome lipid bilayer membranes. The outermost surface coating of hydrophilic polymer chains is effective to provide a liposome with a long blood circulation lifetime in vivo. Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide. The polymers may be employed as homopolymers or as block or random copolymers.

A preferred hydrophilic polymer chain is polyethyleneglycol (PEG), preferably as a PEG chain having a molecular weight between 500-10,000 daltons, more preferably between 500-5,000 daltons, most preferably between 1,000-2,000 daltons. Methoxy or ethoxy-capped analogues of PEG are also preferred hydrophilic polymers, commercially available in a variety of polymer sizes, e.g., 120-20,000 daltons.

Preparation of vesicle-forming lipids derivatized with hydrophilic polymers has been described, for example in U.S. Pat. No. 5,395,619. Preparation of liposomes including such derivatized lipids has also been described, where typically, between 1-20 mole percent of such a derivatized lipid is included in the liposome formulation. It will be appreciated that the hydrophilic polymer may be stably coupled to the lipid, or coupled through an unstable linkage which allows the coated liposomes to shed the coating of polymer chains as they circulate in the bloodstream or in response to a stimulus.

Importantly, liposomes are capable of carrying a therapeutic compound within the aqueous central core of the liposome and between the liposome lipid bilayer, or within the lipid bilayer membrane. Entrapped as used herein is intended to include encapsulation of an agent in the aqueous core and aqueous spaces of liposomes as well as entrapment of an agent in the lipid bilayer(s) of the liposomes.

Antitumor compounds contemplated for use in the invention include, but are not limited to, plant alkaloids, such as vincristine, vinblastine and etoposide; anthracycline antibiotics including doxorubicin, epirubicin, daunorubicin; fluorouracil; antibiotics including bleomycin, mitomycin, plicamycin, dactinomycin; topoisomerase inhibitors, such as camptothecin and its analogues; and platinum compounds, including cisplatin and its analogues, such as carboplatin. Other traditional chemotherapeutic agents suitable for use are known to those of skill in the art and include aminoglutethimide, asparaginase, busulfan, chlorambucil, cyclophosphamide, cytarabine, dacarbazine, estramustine phosphate sodium, floxuridine, fluorouracil (5-FU), flutamide, hydroxyurea (hydroxycarbamide), ifosfamide, interferon Alfa-2a, Alfa-2b, leuprolide acetate (LHRH-releasing factor analogue), lomustine (CCNU), mechlorethamine HCl (nitrogen mustard), melphalan, mercaptopurine, mesna, methotrexate (MTX), mitomycin, mitotane, mitoxantrone, octreotide, procarbazine, streptozocin, tamoxifene, thioguanine, thiotepa, amsacrine (m-AMSA), azacitidine, erythropoietin, hexamethylmelamine (HMM), interleukin 2, mitoguazone (methyl-GAG; methyl glyoxal bis-guanylhydrazone; MGBG), pentostatin, semustine (methyl-CCNU), teniposide (VM-26) and vindesine sulfate.

In one embodiment of the invention, the liposomes have a size suitable for extravasation into a solid tumor. This is particularly useful where the liposomes also include a surface coating of a hydrophilic polymer chain to extend the blood circulation lifetime of the liposomes. Liposomes remaining in circulation for longer periods of time, e.g., more than about 2-5 hours, are capable of extravasating into tumors and sites of infection, which exhibit compromised leaky vasculature or endothelial barriers. Such liposomes are typically between about 50-200 nm, more preferably between 50-150 nm, most preferably between 70-120 nm.

Procedures for preparing and sizing liposomes are widely known to those of skill in the art, as are various methods for entrapping a selected compound, e.g., passive and remote loading methods.

B. Biological Agent

As discussed above in the background section, biological agents for use in the invention are agents of biological origin which produce cytostatis rather than cytotoxicity, as discussed above. Examples of some preferred biological agents are described below.

1. Anti-HER2 Antibody The HER2 proto-oncogene and its encoded p185^{HER2} (ErbB-2) receptor tyrosine kinase play an important role in the pathogenesis of breast and other cancers. In these cancers, HER2 activity is inappropriate or overexpressed which causes, in some cases, HER2 expression in cells which normally do not express HER2 and/or increased HER2 expression leading to unwanted cell proliferation such as cancer.

The HER2 protein is a member of the class I receptor tyrosine kinase family and is structurally related to EGFR, p108(HER3) and p180(HER4). These receptors share a common molecular architecture and contain two cysteine-rich domains within their cytoplasmic domains and structurally related enzymatic regions within their cytoplasmic domains.

Activation of HER2 protein can be caused by different events such as ligand-stimulated homo-dimerization, ligand-stimulated hetero-dimerization and ligand-independent homo-dimerization. HER2 activity can be assayed by measuring one or more of its activities, which include phosphorylation of HER2, phosphorylation of a HER2 substrate and activation of a HER2 adapter molecule.

In addition to breast cancers, increased HER2 activity or gene expression has been associated with certain types of blood cancers, stomach adenocarcinomas, salivary gland adenocarcinomas, endometrial cancers, non-small cell lung cancer and glioblastomas. Determination as to whether or not a cancer is related to an overactivity of HER2 is readily done by those of skill in the art.

Antibodies directed to the HER2 receptor include the murine antibody Mab 4D5 and its humanized counterpart, anti-p185^{HER2} monoclonal antibody, rhuMAb HER2 (Herceptin).

2. Anti-EGFR Antibody Certain cancers, such as glioma, head, neck, gastric, lung, breast, ovarian, colon and prostate, are characterized by an inappropriate activity of epidermal growth factor receptor (EGFR). Such inappropriate activity includes expression of EGFR in cells with normally do not express EGFR and/or increase EGFR expression leading to unwanted cell proliferation, such as in cancer.

One EGFR antagonist is C225, developed by ImClone Systems Incorporated (New York) which is designed to block the EGF receptor. C225 has been administered at doses ranging from 5 mg/m² to 400 mg/m² with not toxicity. Pharmacologically relevant concentration of C225 are reported at dose levels between about 100 mg/m² to 400 mg/m², more preferably between 100 mg/m² to 200 mg/m².

3. Anti-CD20 Antibody B-cell malignancies, such as B-cell leukemias and lymphomas and multiple myeloma, are largely incurable type of hematological cancers. The disease cells are confined mainly in the vascular compartment and patients often respond initially to conventional chemotherapy but in nearly all cases the disease recurs and becomes refractory to further treatment.

Certain cell receptors are expressed on most B-lineage cells, including malignant cells and normal cells. Some cell epitopes, such as CD19, are exclusively expressed on most B-lineage malignancies and are absent on hematopoietic stem cells in the bone marrow. Thus, it is possible to target the malignant cells and leave the progenitor population intact.

Antibodies for B-cells include anti-CD19 and anti-CD20 antibodies.

4. Angiogenesis Inhibitors Tumor growth and metastasis are angiogenesis dependent. Angiostatin, an internal fragment of plasminogen, is a potent inhibitor of angiogenesis, which selectively inhibits endothelial cell proliferation. Angiostatin produced by a primary Lewis lung carcinoma suppressed the growth of lung metastases (O'Reilly, M. S., et al., Cell, 79:315-328(1994)). It is believed that a primary tumor almost completely suppresses the growth of its remote metastases. However, after tumor removal, the previously dormant metastases neovascularize and grow. When the primary tumor is present, metastatic growth is suppressed by a circulating angiogenesis inhibitor. Serum and urine from tumor-bearing mice, but not from healthy, non-tumor bearing control mice, specifically inhibit endothelial cell proliferation. Other evidence suggests that angiostatin is produced by tumor-infiltrating macrophages whose metalloelastase (MME) expression is stimulated by tumor cell-derived granulocyte-macrophage colony-stimulating factor.

Endogenous murine angiostatin, identified as an internal fragment of plasminogen, blocks neovascularization and growth of experimental primary and metastatic tumors in vivo. A recombinant protein comprising kringles 1-4 of human plasminogen (amino acids 93-470) expressed in Pichia pastoris has physical properties (molecular size, binding to lysine, reactivity with antibody to kringles 1-3) that mimic native angiostatin. This recombinant angiostatin protein inhibits the proliferation of bovine capillary endothelial cells in vitro. Systemic administration of recombinant angiostatin protein at doses of 1.5 mg/kg suppressed the growth of Lewis lung carcinoma-low metastatic phenotype metastases in C57BL/6 mice by greater than 90%; administration of the recombinant protein at doses of 100 mg/kg also suppressed the growth of primary Lewis lung carcinoma-low metastatic phenotype tumors. These findings demonstrate unambiguously that the anti-angiogenic and antitumor activity of endogenous angiostatin resides within kringles 1-4 of plasminogen.

When given systemically, angiostatin potently inhibits tumor growth and can maintain metastatic and primary tumors in a dormant state defined by a balance of proliferation and apoptosis of the tumor cells. Angiostatin may act by inducing focal adhesion kinase activity in endothelial cells leading to a subversion of adhesion plaque formation and inhibition of endothelial cell migration and tube formation.

III. Use of the Method

In the method of the invention, a liposome-entrapped chemotherapeutic drug, such as one of those recited above, is administered to a subject suffering from cancer. A biological agent having interaction at some level with the cancer, for example with cancer cell surface receptors or with surrounding tissue, is also administered to the subject.

A. Administration of Anti-EGFR Antibody

In studies performed in support of the invention, a xenograft of the A431 tumor was implanted subcutaneously in nude mice. Six days after tumor implantation, the mice were randomly grouped for treatment with one of the following regimens: free doxorubicin, an anti-EGFR antibody (C225), liposomal-entrapped doxorubicin; free doxorubicin plus the anti-EGFR antibody, or liposome-entrapped doxorubicin plus the anti-EGFR antibody. The liposomes included a vesicle-forming lipid derivatized with the hydrophilic polymer polyethyleneglycol.

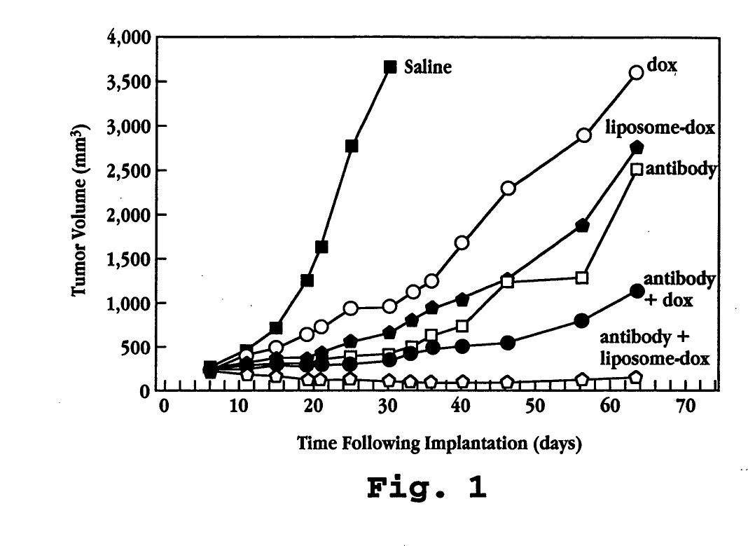

The results are shown in FIG. 1, where the tumor volume, in mm³, is shown as a function of time following tumor implantation, in days. As seen in the figure, mice treated with saline (closed squares) experienced a continuous increase in tumor size. Mice treated with free doxorubicin (open circles), liposome-entrapped doxorubicin (closed diamonds), or anti-EGFR antibody C225 (open squares) fared better than the control mice, with still an increase in tumor size but to a lesser extent. The mice treated with the combined treatments of the therapeutic agent and the biological agent fared best. Surprisingly, the mice treated with the biological agent and the doxorubicin entrapped in liposomes liposome-entrapped doxorubicin (open pentagons) experienced a reduction in tumor volume. In comparison, mice treated with the free doxorubicin and the antibody still had an increase in tumor size.

Table 1 tabulates the size of the tumor at 40 and 60 days following tumor implantation.

TABLE 1
Tumor Volume After Treatment with the Regimens of FIG. 1.

| | Tumor Volume | Tumor Volume |
|---|---|---|
| | at 40 days | at 60 days |
| Treatment Regimen | (mm³) | (mm³) |
| doxorubicin | 1700 | 3650 |
| Liposome-entrapped | 1100 | 2800 |
| doxorubicin | | |
| anti-EGFR antibody | 750 | 2500 |
| doxorubicin + anti-EGFR | 450 | 1150 |
| antibody | | |
| liposome-entrapped | 100 | 125 |
| doxorubicin + anti-EGFR | | |
| antibody | | |

Administration of free doxorubicin in combination with the biological agent C225, the anti-EGFR antibody, achieved a 3.8 fold reduction in tumor volume at 40 days (1700/450) and a 3.2 fold reduction in tumor volume (3650/1150) at 60 days. Entrapping the doxorubicin in liposomes was effective to improve the tumor reduction by 1.5 fold at 40 days (1700/1100) and by 1.3 fold at 60 days (3650/2800). Given these improvements, at most one would expect that administration of the drug in liposome-entrapped form in combination with the biological agent would yield a 4.9 fold improvement (1.4+3.5). Surprisingly, the improvement achieved by administering the drug in liposome-entrapped form in combination with the biological agent is better than 10 fold. At 40 days post tumor implantation, the tumor volume of animals treated with liposome-entrapped doxorubicin was 1100 mm³. Animals treated with the combined therapy of the liposome-entrapped doxorubicin plus the anti-EGFR antibody at the same time point had a tumor volume of 100 mm³. This is an 11 fold reduction in tumor volume, considerably greater than the 4.9 fold reduction predicted. The reduction in tumor volume at 60 days is more pronounced, with a 22 fold reduction in tumor volume for animals treated with the combined therapy of the liposome-entrapped doxorubicin plus the anti-EGFR antibody relative to the animals treated with liposome-entrapped doxorubicin alone (2800/125).

B. Administration of Anti-HER2 Monoclonal Antibody

1. In Combination with Doxorubicin In another series of studies performed in support of the invention the biological agent anti-HER2 monoclonal antibody was administered in combination with free and liposome-entrapped doxorubicin to tumor-bearing mice. As detailed in Example 1, three human breast cancer xenograft models that over-express HER2 were used in the studies. BT474 cells express very high levels of HER2 and MDA453 cells express moderate levels of HER2 (Benz, C. et al., Breast Cancer Res., 24:85-95 (1992)). The primary human breast cancer B585 also expresses high HER2 levels.

In study numbers 1, 2 and 3 immune-deficient mice were inoculated by subcutaneous injection into the dorsum with 5 million BT474 cells in 0.1 mL added to an equal volume of Matrigel for an inoculation volume of 0.2 mL. The inoculated mice were divided into six treatment groups of 12 mice each, prior to inoculation. Treatment was initiated when the average tumor volume was 100 mm³, which occurred approximately 7-10 days after inoculation.

In study number 1, the 12 tumor-bearing mice in each of the six treatment groups ((1)-(6)) were treated as summarized in Table 2.

TABLE 2
Study No. 1 - Treatment and Dosing Regimen
for Mice bearing BT474 tumors

| Treatment | Dose | No. Mice |
|---|---|---|
| saline | Max. vol. once/week x3 | 12 |
| doxorubicin HCl | 4 mg/kg once/week x3 | 12 |
| liposome-entrapped | 3 mg/kg once/week x3 | 12 |
| doxorubicin | | |
| anti-HER2 antibody | 10 mg/kg twice/week x6 | 12 |
| doxorubicin HCl + anti- | 4 mg/kg once/week x3 + | 12 |
| HER2 antibody | 10 mg/kg twice/week x6 | |
| liposome-entrapped | 3 mg/kg once/week x3 + | 12 |
| doxorubicin + anti- | 10 mg/kg twice/week x6 | |
| HER2 antibody | | |

Saline, doxorubicin and liposome-entrapped doxorubicin were administered by intravenous injection for three treatments. The anti-HER2 antibody, Herceptin, was administered by intraperitoneal injection twice weekly for a total of six treatments. The animals were monitored daily and the tumors were measured in three dimensions twice weekly over the study period.

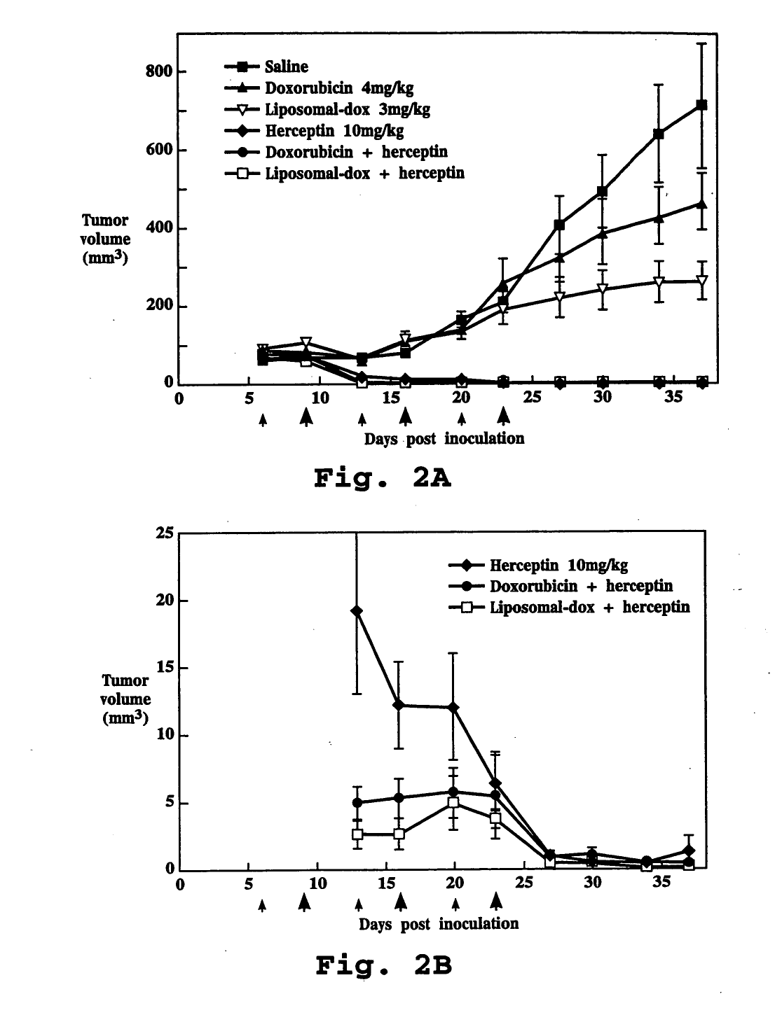

The results are shown in FIGS. 2A-2B. FIG. 2A shows tumor volume as a function of day post inoculation for each of the treatment groups. The large arrowheads along the x-axis indicate administration of drug plus antibody and the smaller arrowheads indicate administration of antibody only. As seen in the figure, the animals treated with saline (closed squares), doxorubicin (closed triangles) and liposome-entrapped doxorubicin (inverted triangles) experienced continual tumor growth over the 3 week treatment period. The animals treated with anti-HER2 antibody (closed diamonds) and with the combination treatments experienced a decrease in tumor volume.

FIG. 2B shows the data of FIG. 2A for the animals treated with anti-HER2 antibody (closed diamonds), doxorubicin+antibody (closed circles) and liposome-entrapped doxorubicin+antibody (open squares). In this study, the anti-HER2 antibody was administered at a dose effective to eliminate tumor growth, making it difficult to differentiate between the combination treatments. While the anti-HER2 antibody eliminates tumor growth, once administration of the antibody ceases, the tumor begins to grow.

Another study was performed using the same tumor model but with a reduced dosage of anti-HER2 antibody. The dosing regimen for Study Number 2 is summarized in Table 3.

TABLE 3
Study No. 2 - Treatment and Dosing Regimen
for Mice bearing BT474 tumors

| Treatment | Dose | No. Mice |
|---|---|---|
| saline | max. vol. once/week x3 | 12 |
| doxorubicin HCl | 5 mg/kg once/week x3 | 12 |
| liposome-entrapped | 5 mg/kg once/week x3 | 12 |
| doxorubicin | | |
| anti-HER2 antibody | 3 mg/kg twice/week x6 | 12 |
| doxorubicin HCl + anti- | 5 mg/kg once/week x3 + 3 mg/kg | 12 |
| HER2 antibody | twice/week x6 | |
| liposome-entrapped | 5 mg/kg once/week x3 + 3 mg/kg | 12 |
| doxorubicin + anti- | twice/week x6 | |
| HER2 antibody | | |

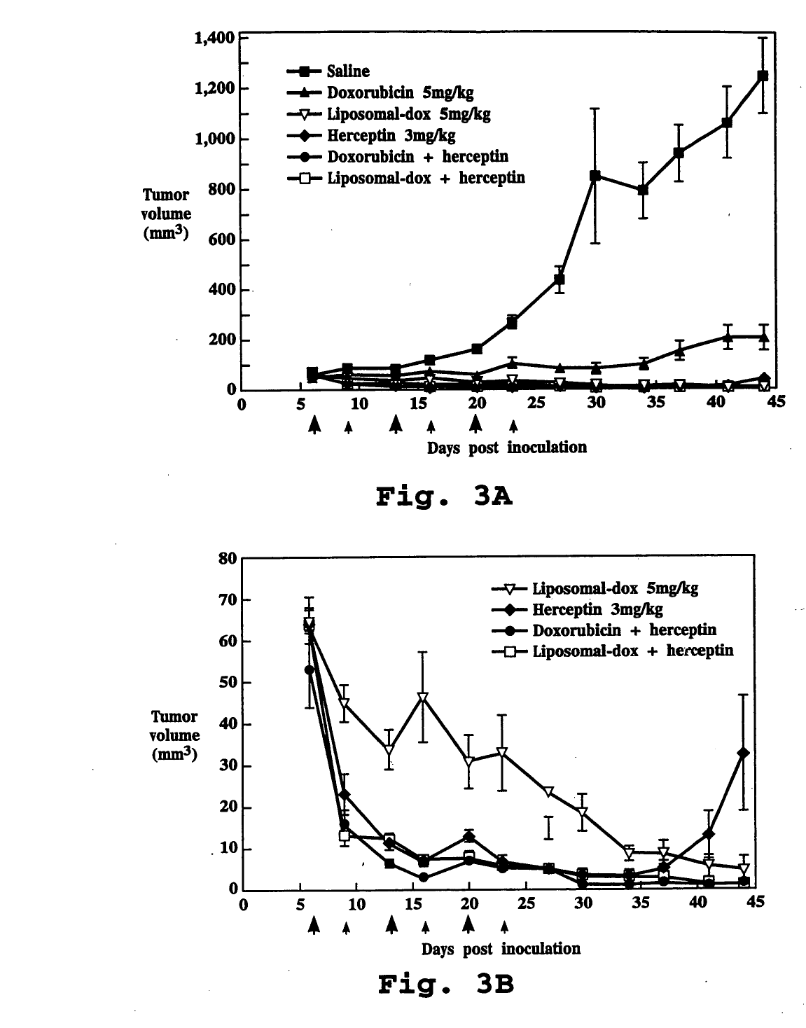

The treatments were administered as described above with respect to Study Number 1. The results after a 3 week treatment period are shown in FIGS. 3A-3B. In FIG. 3A, it can be seen that the untreated animals (saline, closed squares) fared poorly. The animals receiving free doxorubicin (closed triangles) had reduced tumor volume relative to the control animals. The animals receiving liposome-entrapped doxorubicin (inverted triangles), anti-HER2 antibody (diamonds), and the combination treatments are best seen in FIG. 3B. The animals treated with the combination treatments of doxorubicin plus antibody (closed circles) and liposome-entrapped doxorubicin plus antibody (open squares) had a decrease in tumor volume that was statistically different from the animals treated with the antibody alone (see Table 7 in Example 1). The data suggests that the combination treatment is more effective in reducing the tumor than the antibody alone, however, as with the data in FIGS. 2A-2B, it is still not possible to discriminate between the combination treatments.

Study No. 3 was conducted using the same tumor model but with a further refinement of the dosing regimen. In this third study, the test animals were treated as set forth in Table 4.

TABLE 4
Study No. 3 - Treatment and Dosing Regimen for
Mice bearing BT474 tumors

| Treatment | Dose | No. Mice |
|---|---|---|
| saline | max. vol. once/week x3 | 12 |
| doxorubicin HCl | 3 mg/kg once/week x3 | 12 |
| liposome-entrapped | 3 mg/kg once/week x3 | 12 |
| doxorubicin | | |
| anti-HER2 antibody | 1 mg/kg twice/week x6 | 12 |
| doxorubicin HCl + anti- | 3 mg/kg once/week x3 + 1 mg/kg | 12 |
| HER2 antibody | twice/week x6 | |
| liposome-entrapped | 3 mg/kg once/week x3 + 1 mg/kg | 12 |
| doxorubicin + anti- | twice/week x6 | |
| HER2 antibody | | |

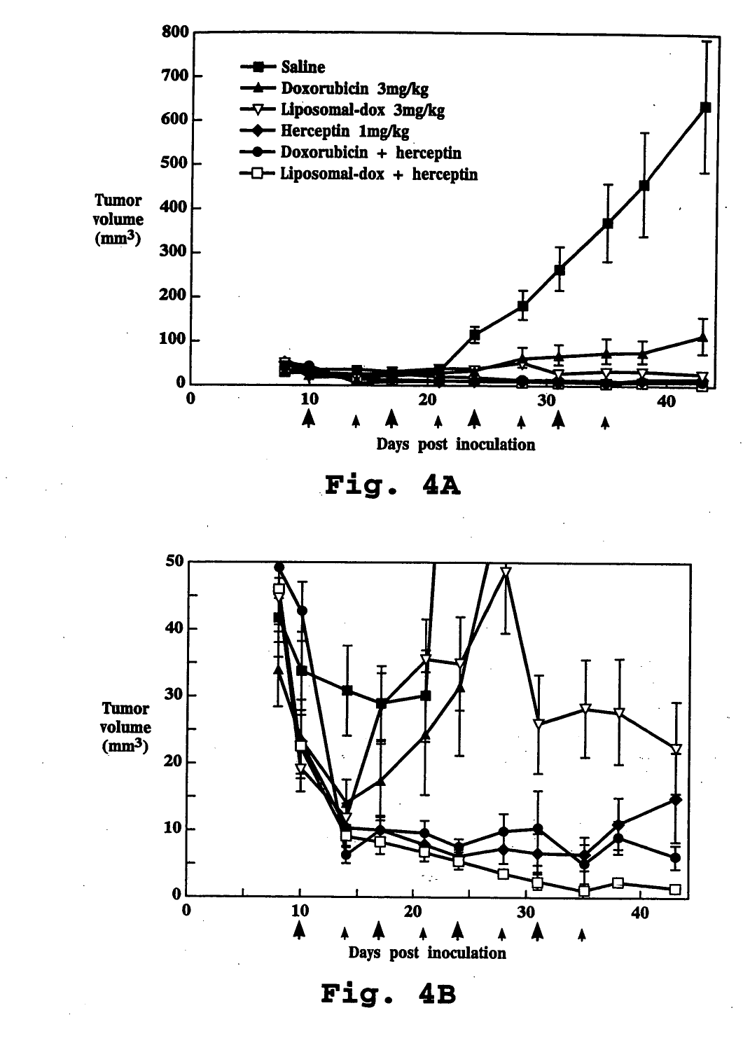

The animals were dosed as described above, with the saline and chemotherapeutic agent administered intravenously once per week and the biological agent administered intraperitoneally twice per week. Tumor volumes were measured regularly over the 3 week test period and the results are shown in FIGS. 4A-4B.

As seen in FIG. 4A, left untreated the tumor continually increases, as evidenced by the saline treated animals (closed squares). Animals treated with a chemotherapeutic agent, doxorubicin (closed triangles) or liposome-entrapped doxorubicin (inverted triangles) fared better than the untreated animals. The results for the animals treated with the antibody alone (diamonds), with free doxorubicin plus antibody (closed circles) and with liposome-entrapped doxorubicin plus antibody (open squares) are seen best in FIG. 4B. At this dosing level, it is possible to discriminate between the combination treatments. The data shows that the animals treated with liposome-entrapped doxorubicin plus anti-HER2 antibody had the greatest tumor reduction. Table 7 in Example 1 sets forth the log growth rate for this study, and indicates that the decrease in tumor growth rate for animals treated with liposome-entrapped doxorubicin plus anti-HER2 antibody was statistically significant when compared to animals treated with free doxorubicin plus anti-HER2 antibody or with the anti-HER2 antibody alone.

Another study, Study No. 4, was conducted using MDA453 xenografts, following the methodology set forth in Example 1. The tumor-bearing animals were treated as set forth in Table 5.

TABLE 5
Study No. 4 - Treatment and Dosing Regimen
for Mice bearing MDA453 tumors

| Treatment | Dose | No. Mice |
|---|---|---|
| saline | max. vol. once/week x3 | 12 |
| doxorubicin HCl | 5 mg/kg once/week x3 | 12 |
| liposome-entrapped | 5 mg/kg once/week x3 | 12 |
| doxorubicin | | |
| anti-HER2 antibody | 5 mg/kg twice/week x6 | 12 |
| doxorubicin HCl + anti- | 5 mg/kg once/week x3 + 5 mg/kg | 12 |
| HER2 antibody | twice/week x6 | |
| liposome-entrapped | 5 mg/kg once/week x3 + 5 mg/kg | 12 |
| doxorubicin + anti- | twice/week x6 | |
| HER2 antibody | | |

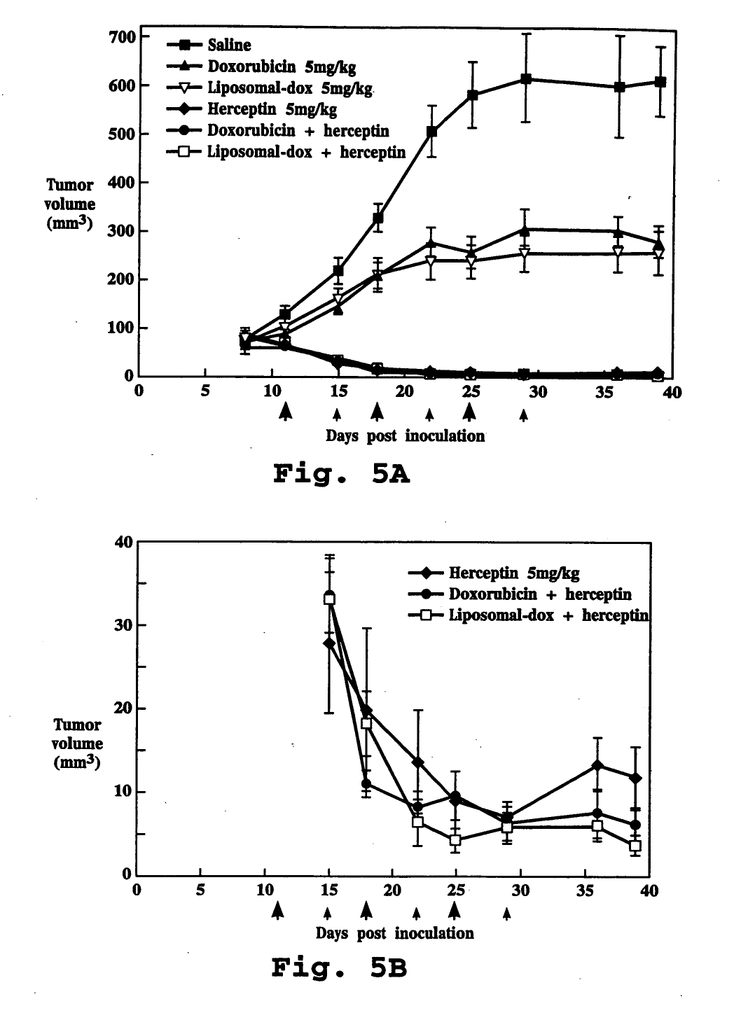

The animals were dosed as described above, with the saline and chemotherapeutic agent administered intravenously once per week and the biological agent administered intraperitoneally twice per week. Tumor volumes were measured regularly over the 3 week test period and the results are shown in FIGS. 5A-5B.

The data in FIG. 5A shows that the animals receiving no biological agent, e.g., saline-treated (closed squares), doxorubicin-treated (closed triangles) and liposome-entrapped doxorubicin-treated (inverted triangles) had a continual increase in tumor volume of the MDA453 xenograft over the test period. The animals receiving the biological agent fared better, with those animals treated with the liposome-entrapped doxorubicin plus anti-HER2 antibody (open squares) have the most significant reduction in tumor volume (also see Table 7 in Example 1).

The primary human breast cancer B585 express high levels of HER2. In Study No. 5, mice were inoculated with passaged B585 tumor tissue. At 16 days post inoculation, when tumors were established, the mice were treated as set forth in Table 6.

TABLE 6
Study No. 5 - Treatment and Dosing Regimen
for Mice bearing B585 tumors

| Treatment | Dose | No. Mice |
|---|---|---|
| saline | max. vol. once/week x3 | 12 |
| doxorubicin HCl | 4 mg/kg once/week x3 | 12 |
| liposome-entrapped | 4 mg/kg once/week x3 | 12 |
| doxorubicin | | |
| anti-HER2 antibody | 3 mg/kg twice/week x6 | 12 |
| doxorubicin HCl + anti- | 4 mg/kg once/week x3 + 3 mg/kg | 12 |
| HER2 antibody | twice/week x6 | |
| liposome-entrapped | 4 mg/kg once/week x3 + 3 mg/kg | 12 |
| doxorubicin + anti- | twice/week x6 | |
| HER2 antibody | | |

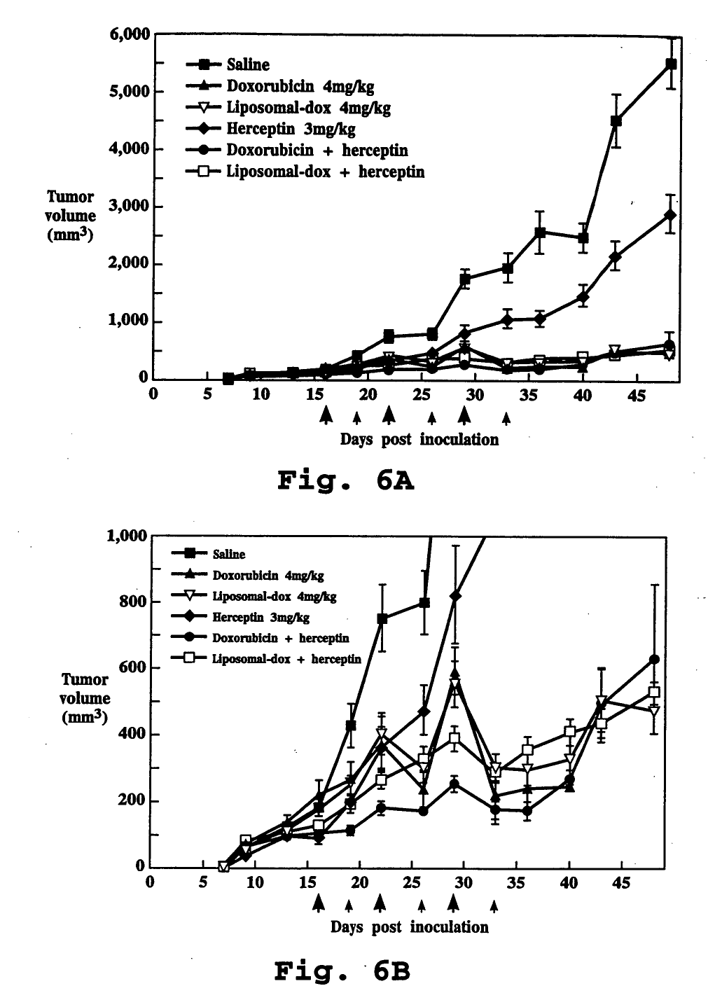

The animals were dosed as described above, with the saline and chemotherapeutic agent administered intravenously once per week and the biological agent administered intraperitoneally twice per week. Tumor volumes were measured regularly over the 3 week test period and the results are shown in FIGS. 6A-6B.

As seen in FIG. 6A, animals treated with anit-HER2 antibody alone (diamonds) experienced continuous tumor growth. While the B5b5 tumor cells express high levels of HER2, the cells appear to be resistant to the anti-HER2 antibody. The combination treatments of chemotherapeutic agent plus antibody (closed circles, open squares) provided an improved treatment efficacy relative to the antibody alone, but were not statistically better than the animals treated with liposome-entrapped doxorubicin alone (inverted triangles, see Table 7, Example 1).

This comparative study no. 5 indicates that a biologic agent having activity against the tumor cells is required to obtain the synergistic effect in accord with the invention.

2. In Combination with Cisplatin Like many chemotherapeutic agents, cisplatin can be toxic at the dosages required for effective cancer therapy. Entrapping cisplatin in liposomes, and in particular in liposomes having a surface coating of polyethylene glycol to extend their blood circulation lifetime, has been shown to improve the antitumor efficacy relative to free cisplatin (U.S. Pat. No. 5,945,122; Newman M. et al., Cancer Chemother. Pharmacol., 43:1-7 (1999)). These same studies demonstrate that cisplatin entrapped in liposomes has reduced renal accumulation and toxicity compared to free cisplatin.

Studies were performed in support of the invention to demonstrate the synergistic antitumor activity of a liposome-entrapped therapeutic agent, as exemplified by cisplatin, and a biological agent, as exemplified by anti-HER2 antibody. In these studies, two models of human breast cancer were used, and the antitumor activity of the combination therapy in accord with the invention was compared to the activity of the anti-HER2 antibody alone, of free cisplatin and to free cisplatin plus antibody.

As described in Example 2, BT474 cells or MDA453 cells were used to inoculate immune-deficient mice. As noted above, BT474 cells express very high levels of HER2 while MDA453 cells express a moderate level of HER2. The inoculated mice were treated according to the regimens now to be described with respect to Study Numbers 6-8.

In Study No. 6, mice were inoculated with BT474 cells. Six days after inoculation, when a tumor was established, the animals were treated as indicated in Table 8.

TABLE 8
Study No. 6 - Treatment and Dosing Regimen
for Mice bearing BT474 tumors

| Treatment | Dose | No. Mice |
|---|---|---|
| saline | 0.1 ml once/week x3 | 12 |
| cisplatin | 6 mg/kg once/week x3 | 12 |
| liposome-entrapped | 6 mg/kg once/week x3 | 12 |
| cisplatin | | |
| anti-HER2 antibody | 3 mg/kg twice/week x6 | 12 |
| cisplatin + anti- | 6 mg/kg once/week x3 + | 12 |
| HER2 antibody | 3 mg/kg twice/week x6 | |
| liposome-entrapped | 6 mg/kg once/week x3 + | 12 |
| cisplatin + anti- | 3 mg/kg twice/week x6 | |
| HER2 antibody | | |

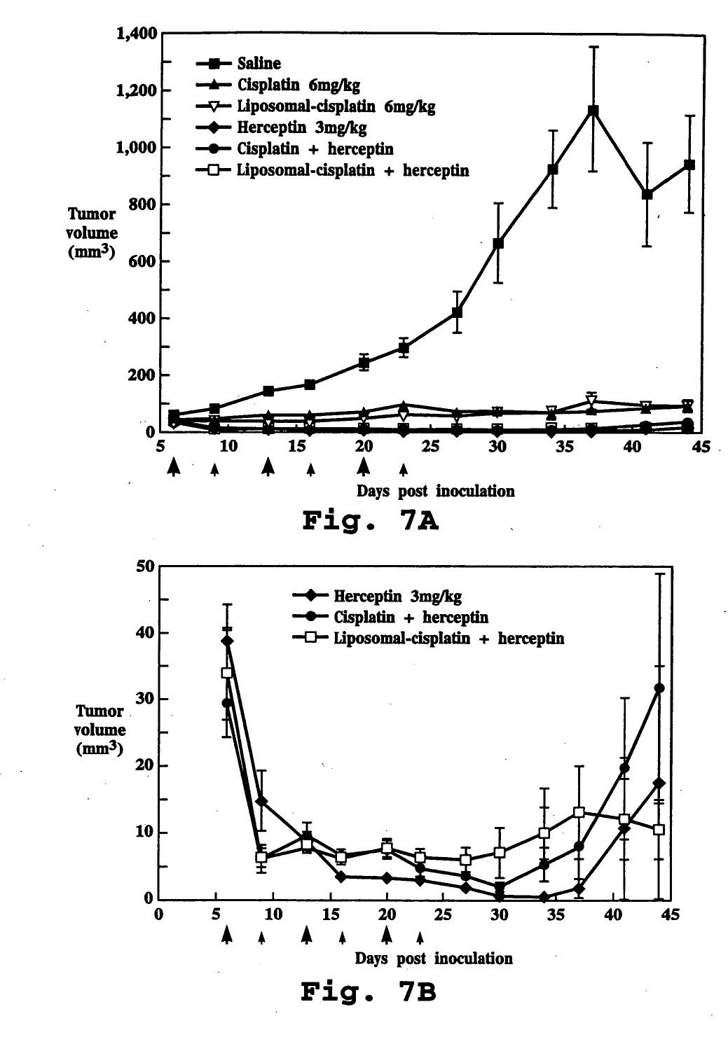

The animals were dosed as described in Example 2, with the saline and chemotherapeutic agent administered intravenously once per week and the biological agent administered intraperitoneally twice per week. Tumor volumes were measured regularly over the 3 week test period and the results are shown in FIGS. 7A-7B.

As seen in FIG. 7A, left untreated, the tumor grows continuously, as evidenced by the animals treated with saline (closed squares). The animals treated with free cisplatin (closed triangles) or with liposome-entrapped cisplatin (inverted triangles) fared considerably better than the untreated animals. The animals receiving the anti-HER2 antibody alone (diamonds) or in combination with cisplatin, either in free form (closed circles) or in liposome-entrapped form (open squares) had little to no tumor growth. As seen best in FIG. 7B, the animals treated with liposome-entrapped cisplatin plus anti-HER2 antibody had a statistically significant lower tumor growth rate than the animals treated with cisplatin in free form plus the antibody (see also Table 11 in Example 2).

In Study No. 7, the dosage of the antibody was reduced to better differentiate between the treatment regimens. Animals inoculated with BT474 tumor cells were treated as set forth in Table 9.

TABLE 9
Study No. 7 - Treatment and Dosing Regimen
for Mice bearing BT474 tumors

| Treatment | Dose | No. Mice |
|---|---|---|
| saline | 0.1 ml once/week x3 | 12 |
| cisplatin | 4 mg/kg once/week x3 | 12 |
| liposome-entrapped | 4 mg/kg once/week x3 | 12 |
| cisplatin | | |
| anti-HER2 antibody | 0.5 mg/kg twice/week x6 | 12 |
| cisplatin + anti- | 4 mg/kg once/week x3 + 0.5 mg/kg | 12 |
| HER2 antibody | twice/week x6 | |
| liposome-entrapped | 4 mg/kg once/week x3 + 0.5 mg/kg | 12 |
| cisplatin + anti- | twice/week x6 | |
| HER2 antibody | | |

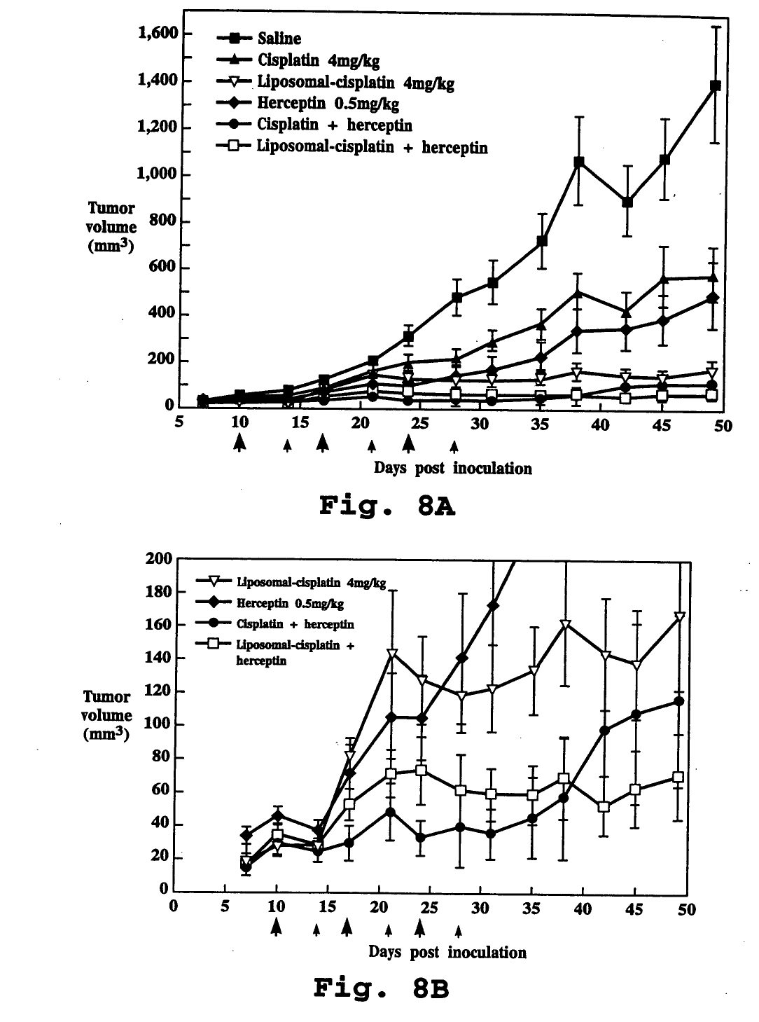

The treatments were administered to the animals as described above and the results are shown in FIG. 8. The large arrowheads along the x-axis indicate administration of drug plus antibody and the smaller arrows indicate administration of antibody only. As seen in the figure, the animals treated with saline (closed squares), cisplatin (closed triangles) and anti-HER2 antibody (diamonds) experienced continual tumor growth over the 3 week treatment period. The animals treated with liposome-entrapped cisplatin (inverted triangles) and with the combination treatments experienced little increase in tumor volume. The treatment of liposome-entrapped cisplatin plus antibody provided the most efficacious therapy.

Another study was performed using the MDA453 xenograft model. Immune deficient mice were inoculated with MDA453 tumor cells-as described in Example 2. Eight days after inoculation, the mice were treated with the therapies set forth in Table 10.

TABLE 10
Study No. 8 - Treatment and Dosing Regimen
for Mice bearing MDA453 tumors

| Treatment | Dose | No. Mice |
|---|---|---|
| saline | 0.1 ml once/week x3 | 12 |
| cisplatin | 4 mg/kg once/week x3 | 12 |
| liposome-entrapped | 4 mg/kg once/week x3 | 12 |
| cisplatin | | |
| anti-HER2 antibody | 1 mg/kg twice/week x6 | 12 |
| cisplatin + anti- | 4 mg/kg once/week x3 + 1 mg/kg | 12 |
| HER2 antibody | twice/week x6 | |
| liposome-entrapped | 4 mg/kg once/week x3 + 1 mg/kg | 12 |
| cisplatin + anti- | twice/week x6 | |
| HER2 antibody | | |

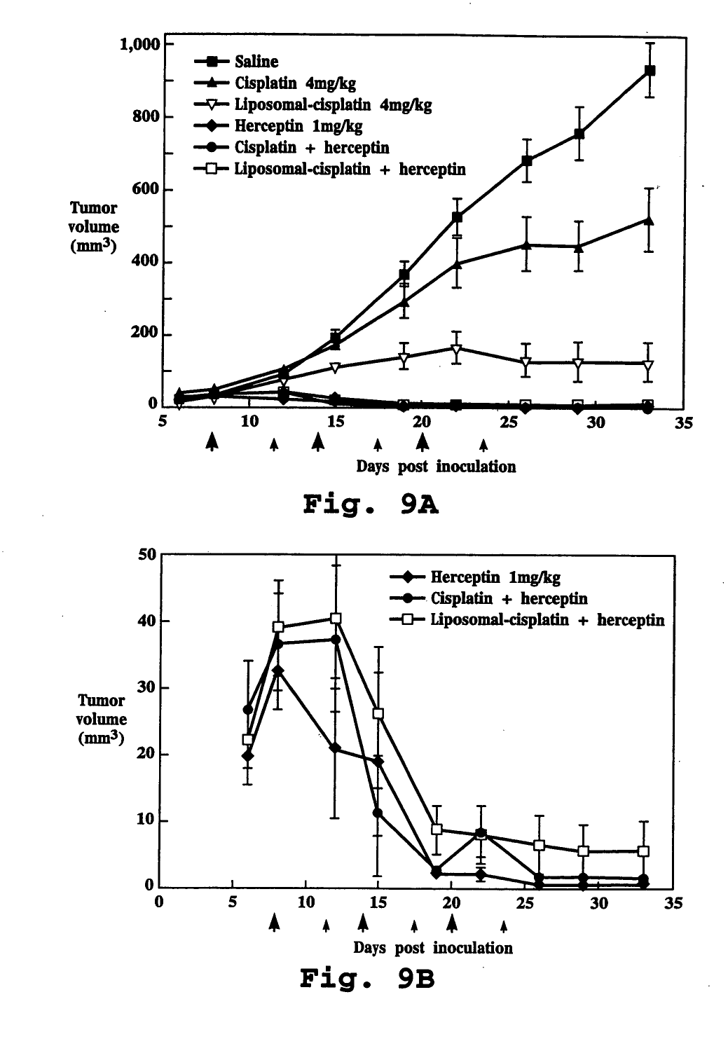

Following the dosing regimen described above, the therapies were administered to the test animals. Tumor volume over the course of the experiment is shown in FIGS. 9A-9B, where the large arrowheads along the x-axis indicate administration of drug plus antibody and the smaller arrows indicate administration of antibody only.

As seen in FIG. 9A, the animals treated with saline (closed squares), free cisplatin (closed triangles) experienced continual tumor growth over the 3 week treatment period. The animals treated with liposome-entrapped cisplatin (inverted triangles) experienced a reduction in tumor volume after about day 22. The results for animals treated with and anti-HER2 antibody (diamonds) and with the combination treatments are best seen in FIG. 9B. For this tumor model at these dosages, no statistical difference between the three test groups receiving the antibody was observed.

Studies 6-8 demonstrate that the combination of liposome-entrapped cisplatin and anti-HER2 antibody had significant antitumor efficacy and offers a potentially less toxic treatment regimen for cancer patients.

More generally, the results discussed above with respect to FIGS. 1-5 and 7-9 indicate that entrapping a chemotherapeutic drug in liposomes and administering the liposome-entrapped agent in combination with a biological agent is effective to potentiate the response of the chemotherapeutic drug. It will be appreciated that the biological agent can be administered concurrently with the liposome-entrapped compound or after administration of the liposome-entrapped compound. For example, the liposome-entrapped agent and the biological agent can be administered simultaneously as a bolus injection or as a continuous infusion. Alternatively, the liposome-entrapped compound can be administered first, as an injection or slow infusion, and allowed to circulation and distribute in the patient. The biological agent is then administered as a bolus or slow infusion. This latter regimen is particularly suitable for use with long-circulating liposomes, e.g., liposomes having a surface coating of hydrophilic polymer chains.

In a preferred embodiment, the therapeutic agent is administered to the patient entrapped in long-circulating liposomes. The liposomes are allowed to distribute and to extravasate into the tumor site, typically this process takes between 5-24 hours. The biological agent is administered during this time period to enhance the effect of the therapeutic agent.

Cancers contemplated for treatment by the method of the invention include, but are not limited to acute lymphocytic leukemia (ALL), acute myelogenous leukemia (AML), breast Cancer, choriocarcinoma, embryonal rhabdomyosarcoma, Ewing's sarcoma, hairy cell leukemia, Hodgkin's disease, lung (small cell, oat cell), Non-Hodgkin's lymphoma, Burkitt's lymphoma, diffuse large cell lymphoma, osteogenic sarcoma, testicular, Wilm's tumor, adrenocortical carcinoma, bladder, brain glioblastoma, medulloblastoma, cervix, chronic lymphocytic leukemia, chronic myelogenous leukemia (CML), endometrial, gastric, head and neck, squamous cell, islet cell carcinoma, Kaposi's sarcoma (AIDS-related), mycosis fungoides, myeloma, neuroblastoma, Non-Hodgkin's lymphoma, follicular lymphoma, colorectal, liver, lung (non-small cell), melanoma, pancreatic, and renal.

Based on the results shown in FIGS. 1, 4A-4B, 5A-5B and 7A-7B it is clear that the combined treatment of the liposome-entrapped anticancer agent plus the biological agent potentiates the activity of the anticancer agent. Thus, the invention includes, in another aspect, a method of treating a subject for a cancer derived from over-expression of receptor by administering to the subject a sub-therapeutic amount of the anticancer agent entrapped in long-circulating liposomes e.g., liposomes with a surface coating of hydrophilic polymer chains, in conjunction with the biological agent. For example, liposome-entrapped doxorubicin is typically administered alone at a dose effective to achieve a therapeutic response, which, for tumor therapy, would be evidenced by a reduction in tumor volume. In the method of the invention, the liposome-entrapped doxorubicin is administered at a dosage less than the dose effective to achieve the reduction in tumor volume. A biological agent having activity for the target cancer tissue or cells is administered with the liposome-entrapped compound. Specific examples include administration of liposome-entrapped doxorubicin in combination with an anti-HER2 antibody or administration of liposome-entrapped doxorubicin in combination with anti-EGFR antibody. Another example is administration of a sub-therapeutic dose of liposome-entrapped doxorubicin or another anthracycline antibiotic along with a dose of a biological agent having activity with surface receptors associated with B-cells, for treatment for example, of a B-cell-derived lymphoma. The dose of the biological agent can be readily determined by those of skill in the art using typical methodologies to determine suitable dosing ranges.

### IV. EXAMPLES

The following examples illustrate the method of the invention and are in no way intended to limit its scope.

### Example 1

### Anti-HER2 Antibody Administered with Doxorubicin

A. Test Formulations

1. Saline: Normal saline, at the maximal volume used to treat any treatment group, was used to treat negative control animals.

2. Liposome Formulation: Doxorubicin entrapped in liposomes having a surface coating of polyethylene glycol chains (DOXIL®, doxorubicin HCl liposome injection, SEQUUS Pharmaceuticals, Menlo Park, Calif.), was used. The liposome composition (% mol ratio) was hydrogenated soybean phosphatidylcholine (56.2), cholesterol (38.3) and methoxypolyethylene glycol-2000-distearoyl-phosphatidylethanolamine (5.3). Doxorubicin was encapsulated in liposomes at a drug:lipid ratio of approximately 150 mg/mmol lipid in the presence of 250 mM ammonium sulfate. More than 95% of drug was in encapsulated form. The liposomes had an average diameter of 90 nm. The liposome formulation was supplied at a concentration of 2 mg/mL doxorubicin, and all doses were measured and expressed on the basis of doxorubicin content.

3. Free Doxorubicin: Doxorubicin (Bedford Laboratories, Bedford, Ohio) was used to treat positive control groups. Doxorubicin HCl was diluted to appropriate concentrations for injection in normal saline (0.9% NaCl) immediately prior to injection.

4. Anti-HER2 Antibody: Herceptine (Genentech, Inc., So. San Francisco, Calif.) was used to treat experimental treatment groups. Appropriate dilutions were made in buffer supplied by the manufacturer prior to injection.

B. Tumor Lines

Three separate HER2-overexpressing human breast cancer xenograft models were utilized: BT474, MDA453 and B585. BT474 cells express very high levels of HER2 (20×receptor over-expression relative to MCF7 cells, Benz, C. et al., Breast Cancer Res., 24:85-95 (1992)), while MDA453 cells express a moderate level (7× receptor over-expression relative to MCF7 cells, Benz, C. et al., Breast Cancer Res., 24:85-95 (1992)). The primary human breast cancer, B585, also expresses high levels of HER2, based on immunohistochemical staining with the antibody 4D5.

1. BT474 and MDA453 Cells: The BT474 and MDA453 tumors were each inoculated from cultured cells in exponential growth. Cells were trypsinized, collected and washed in media. Cells were counted by hemacytometer, spun down and resuspended in media at 50 million cells per milliliter of media. Resuspended cells were chilled, then mixed with an equal volume of Matrigel. Cells (5 million cells per 0.2 ml injection volume, continuously mixed) were drawn into individual, chilled syringes for subcutaneous injection into the dorsum of each animal.

2. B585 Human Cells: The B585 primary human breast xenografts were inoculated from tumor tissue harvested from tumor bearing animals. Tumors were minced with scissors and resuspended in media. Resuspended cells were chilled, then mixed with an equal volume of Matrigel. Cells (5 million cells per 0.2 ml injection volume, continuously mixed) were drawn into individual, chilled syringes for subcutaneous injection into the dorsum of each animal.

C. Animals

Immune deficient mice were used in all experiments. Study numbers 1, 2, 3 and 4 were conducted in homozygous nude female mice. Study number 5 was conducted in homozygous ICR scid female mice. All mice were obtained at the age of four weeks and acclimated for a minimum of 1 week prior to tumor inoculation. All mice were ear tagged for individual identification during acclimation. For each study, 75 mice were inoculated with tumors to supply 72 study animals at treatment.

Animals were housed in a Thoren Microisolator Caging System in a HEPA filtered biocontainment suite. Animals were acclimated to the laboratory conditions for at least 1 week. Only healthy animals were assigned to the study.

Autoclaved and acidified water and gamma irradiated standard rodent diet were supplied ad libitum to all animals throughout the study.

All animals were observed at least daily for general well-being. Animals were weighed prior to inoculation of tumors and at least weekly thereafter. Tumors were measured twice weekly throughout the experiment, beginning 6-8 days after tumor inoculation. Tumor measurements were made by digital caliper in 3 dimensions and the volume recorded as one-half the product of these measurements. Any animal observed to have 15% or greater weight loss from the initial starting weight was immediately euthanized by inhalation of 100% carbon dioxide. Any animal observed to have greater than 4,000 mm³ tumor volume was immediately euthanized by inhalation of 100% carbon dioxide.

D. Dosing Regimen

Animals were divided into 6 treatment groups prior to inoculation of tumors. Treatment groups received: (1) 0.1 ml saline once weekly; (2) 3 to 5 mg/kg doxorubicin HCl once weekly; (3) 3 to 5 mg/kg DOXIL once weekly; (4) 1 to 10 mg/kg Herceptin twice weekly; (5) 3 to 5 mg/kg doxorubicin weekly and 1 to 10 mg/kg Herceptin twice weekly; (6) 3 to 5 mg/kg DOXIL weekly+1 to 10 mg/kg Herceptin twice weekly. Saline, DOXIL and doxorubicin were administered by intravenous injection, Herceptin was administered by intraperitoneal injection. Treatment was initiated when average tumor volume was 100 mm³ (approximately 7-10 days after inoculation). Tumor sizes within each treatment group were compared to confirm similar initial size prior to initiation of treatment.

The dosing regimens are summarized in Tables 2-6 above.

E. Results

Tumor volumes, measured repeatedly throughout the experiments, were used for analysis as correlated information. Since tumor growth over time after treatment was of interest, repeated measurement analysis for each treatment group was performed. All tumor volumes were log transformed, with z=log₁₀(y+1) where y is the calculated tumor volume, thus when y=0, z is meaningful and z=0. Log growth rates for transformed values, z, for each treatment group within each experiment were calculated and compared in linear regression. If the log growth rate is positive for a certain treatment, this means that the tumors still grow even after receiving the drug; if the log growth rate is negative, this means that the tumors begin to shrink after receiving the drug; if the log growth rate is 0, then the tumors stop growing after receiving the drug. The log growth rates for all experiments are given in Table 7. A p-value of 0.05 or less is statically significant.

TABLE 7
Log Growth Rates for Studies 1-5

| | Study | Study | Study | Study | Study |
|---|---|---|---|---|---|
| Drug | No. 1 | No. 2 | No. 3 | No. 4 | No. 5 |
| Saline | 0.031^{a,b} | 0.038^{a,b,c} | 0.040^{a,b,c} | 0.028^{a,b,c} | 0.060^{a} |
| Doxorubicin | 0.027 | 0.010^{a} | 0.011^{a} | 0.020^{a} | 0.052 |
| liposome- | 0.009^{a} | −0.033^{b} | −0.004^{b} | 0.017^{b} | 0.034^{a} |
| entrapped | | | | | |
| doxorubicin | | | | | |
| Herceptin | −0.061^{b} | −0.019^{c,d,e} | −0.020^{c,d} | −0.030^{c,d} | 0.061^{b,c} |
| Doxorubicin + | −0.056 | −0.030^{d} | −0.023^{e} | −0.035 | 0.039^{b} |
| Herceptin | | | | | |
| liposome- | −0.053 | −0.030^{e} | −0.033^{d,e} | −0.043^{d} | 0.032^{c} |
| entrapped | | | | | |
| Dox. + Herceptin | | | | | |

^{a,b,c,d,e}Within each column, growth rates with the same superscript are statistically different (p < 0.05), indicating a significant treatment effect.

### Example 2

### Anti-HER2 Antibody Administered with Cisplatin

A. Test Formulations

1. Saline: Normal saline, at the maximal volume used to treat any treatment group, was used to treat negative control animals.

2. Liposome Formulation: Cisplatin entrapped in liposomes having a surface coating of polyethylene glycol were prepared as described in U.S. Pat. No. 5,945,122, which is incorporated by reference and in Newman M. et al., Cancer Chemother. Pharmacol., 43:1-7 (1999). The liposomal-cisplatin formulation was composed of N-(carbamoyl-methoxypolyethylene glycol 2000)-1,2-disteroyl-sn-glycero-3-phosphatidylethanolamine sodium salt (mPEG-DSPE), hydrogenated soy phosphatidylcholine (HSPE) and cholesterol combined with cisplatin under ethanol injection. The cisplatin is 100% encapsulated in 100 nm average sized liposomes after diafiltration.

3. Free Cisplatin: Cisplatin (Platinol AQ, Bristol Laboratories) was purchased from standard suppliers and reconstituted and maintained according manufacturers recommendations.

4. Anti-HER2 Antibody: Herceptin® (Genentech, Inc., So. San Francisco, Calif.) was used to treat experimental treatment groups. Appropriate dilutions were made in buffer supplied by the manufacturer prior to injection.

B. Tumor Lines

Two separate HER2-overexpressing human breast cancer xenograft models were utilized: BT474 and MDA453. The BT474 or MDA453 tumors were inoculated from cultured cells in exponential growth. Cells were trypsinized, collected and washed in media. Cells were counted by hemacytometer, spun down and resuspended in media at 50 million cells per milliliter of media (5 million cells per 0.1 ml injection volume). Resuspended cells were chilled, then mixed in an equal volume of Matrigel. Cells (0.2 ml, continuously mixed) were drawn into individual, chilled syringes for subcutaneous injection into the dorsum of each animal.

C. Animals

Immune-deficient female mice (NCR.nu/nu, Taconic Farms, Germantown, N.Y.) were obtained from the vendor and acclimated for at least 7 days prior to initiation of experiments. Animals were housed in microisolator caging (Thoren Caging, Hazelton, Pa.) with ad lib gamma irradiated rodent food and autoclaved, acidified water. Lights were set for 12:12 light: dark cycle. All animals received a 0.72 mg estradiol 17β sustained release pellet (Innovative Research of America, Sarasota, Fla.) by subcutaneous injection 24 hours prior to tumor inoculation. Animals were randomized into treatment groups prior to inoculation of tumors.

All animals were observed at least daily for general well being. Animals were weighed prior to inoculation of tumors and at least weekly thereafter. Tumors were measured twice weekly throughout the experiment, beginning 6-8 days after tumor inoculation. Tumor measurements were made by digital caliper in 3 dimensions and the volume recorded as one half the product of these measurements. Any animal observed to have 15% or greater weight loss from the initial starting weight was immediately euthanized. Any animal observed to have greater than 4,000 mm³ tumor volume was immediately euthanized.

D. Dosing Regimen

Animals were divided into 6 treatment groups (12 animals each) prior to inoculation of tumors in all experiments. Treatment groups received: (1) 0.1 ml saline once weekly; (2) 4 to 6 mg/kg nonliposomal cisplatin once weekly (6 mg/kg is MTD); (3) 4 to 6 mg/kg PL-cisplatin once weekly (6 mg/kg is MTD); (4) 0.5 to 3 mg/kg Herceptin twice weekly (no MTD established as antibody does not interact with mouse tissues); (5) 4 to 6 mg/kg nonliposomal cisplatin weekly and 0.5 to 3 mg/kg Herceptin twice weekly; (6) 4 to 6 mg/kg PL-cisplatin weekly+0.5 to 3 mg/kg Herceptin twice weekly.

Saline, cisplatin and PL-cisplatin were administered by intravenous injection; Herceptin was administered by intraperitoneal injection. All treatments were administered for 3 weekly cycles. Treatment was initiated when average tumor volume was 75 mm³ (approximately 6-10 days after inoculation). Tumor sizes within each treatment group were compared to confirm similar initial size prior to initiation of treatment.

The dosing regimens are summarized in Tables 8-10 above.

E. Results

Tumor volumes, measured repeatedly throughout experiments, were used for analysis as correlated information. Since tumor growth over time after treatment was of interest, repeated measurement analysis for each treatment group was performed. Growth rates (slope) for each treatment group within each experiment were calculated and compared in linear regression. If the growth rate was positive for a certain treatment, tumors were still growing even after receiving the drug; if the growth rate is negative, tumors began to shrink after receiving the drug; if the growth rate is 0, tumors stop growing after receiving the drug. The growth rates for all experiments are given in Table 11. A p-value of 0.05 or less is considered significant.

TABLE 11
Growth rate of tumors (slope) from Studies 6-8.

| | Study No. 6 | Study No. 7 | Study No. 8 |
|---|---|---|---|
| Treatment | BT474 | BT474 | MDA453 |
| Saline | 28.44 | 32.58 | 35.82 |
| Cisplatin | 1.02 | 14.35^{b} | 19.84^{e} |
| liposome-entrapped | 1.71 | 3.38^{b} | 4.28^{e} |
| cisplatin | | | |
| anti-HER2 antibody | −0.34 | 10.78^{c,d} | −1.14 |
| Cisplatin + anti-HER2 | 0.06^{a} | 2.14^{c} | −1.37 |
| antibody | | | |
| liposome-entrapped − | −0.37^{a} | 0.92^{d} | −1.25 |
| cisplatin + anti-HER2 | | | |
| antibody | | | |

Comparison of the growth rate (slope) between different treatment groups that are noted by the same superscript letter have significance as follows: ^{a}p = 0.04,^{b-e} p < 0.0001.

Tumor size at specific times after treatment was compared between treated and control groups. Tumor growth inhibition was reported as % T/C which is the tumor size of each treated group divided by the control group, expressed as a percentage.

The National Cancer Institute uses a value of <42% T/C as indicating significant antitumor activity. The results are shown in Table 12.

TABLE 12
Size of treated tumors as a function of size of control tumors¹,
% T/C, at conclusion of each study.

| | Study No. 6 | Study No. 7 | Study No. 8 |
|---|---|---|---|
| Treatment | Day 44 | Day 49 | Day 33 |
| Cisplatin | 10.0 | 41.7 | 56.1 |
| liposome-entrapped | 9.5 | 11.9 | 13.5 |
| cisplatin | | | |
| anti-HER2 antibody | 1.9 | 35.5 | 0.1 |
| Cisplatin + anti-HER2 | 3.3 | 8.3 | 0.2 |
| antibody | | | |
| liposome-entrapped | 1.1 | 5.0 | 0.6 |
| cisplatin + anti-HER2 | | | |
| antibody | | | |

¹control tumors from saline treated animals

Although the invention has been described with respect to particular embodiments, it will be apparent to those skilled in the art that various changes and modifications can be made without departing from the invention.

1-23. (canceled)

24. A method for potentiating the activity of a chemotherapeutic drug administered in combination with a biological agent to a subject suffering from cancer, comprising;
entrapping the chemotherapeutic drug in a liposome, and administering the liposome-entrapped drug with the biological agent in free form,
wherein said administering is effective to produce at least about a 3.8-fold reduction in in vivo tumor volume relative to that provided by administering the chemotherapeutic drug in combination with the biological agent both in free form, the reduction in tumor volume being determined 40 days following implantation of a xenograft tumor in mice.

25. The method of claim 24, wherein the liposome-entrapped drug is an anthracycline antibiotic.

26. The method of claim 25, wherein the anthracycline antibiotic is selected from the group consisting of doxorubicin, daunorubicin, epirubicin, idarubicin and analogs thereof.

27. The method of claim 24, wherein the cancer is characterized by over-activity of a tyrosine kinase receptor and the biological agent is capable of binding to such a receptor.

28. The method of claim 27, wherein the tyrosine kinase receptor is selected from the group consisting of HER2, EGF and PDGF.

29. The method of claim 27, wherein the biological agent is selected from the group consisting of anti-HER2 antibody, anti-EGFR antibody and anti-PDGFR antibody.

30. The method of claim 24, wherein the cancer is derived from a B-cell malignancy and the biological agent is capable of binding to a B-cell surface antigen selected from the group consisting of CD19, CD20, CD22 and CD77.

31. The method of claim 30, wherein the biological agent is selected from the group consisting of anti-CD19 antibodies, anti-CD20 antibodies, anti-CD22 antibodies and anti-CD77 antibodies.

32. The method of claim 24, wherein the biological agent is an anti-angiogenesis agent.

33. The method of claim 32, wherein the anti-angiogenesis agent is selected from the group consisting of angiostatin, endostatin and oncostatin.

34. The method of claim 24, wherein the biological agent is administered concurrently with the liposome-entrapped drug.

35. The method of claim 24, wherein the biological agent is administered after administration of the liposome-entrapped drug.

36. The method of claim 24, wherein the liposomes entrapping the chemotherapeutic drug include a surface coating of hydrophilic polymer chains effective to extend the blood circulation lifetime of the liposomes and said administering includes administering the biological agent after administration of the liposome-entrapped anti-tumor agent.

37. The method of claim 36, wherein the drug is doxorubicin entrapped in liposomes having polyethyleneglycol polymer chains.

38. The method of claim 37, wherein the biological agent is an anti-HER2 antibody for treatment of cancer cells expressing the HER2 receptor.

39. The method of claim 37, wherein the biological agent is an anti-CD20 antibody for treatment of a B-cell lymphoma.

40. The method of claim 24, wherein said administering is effective to produce at least about a 4.5-fold reduction in in vivo tumor volume relative to that provided by administering the chemotherapeutic drug in combination with the biological agent both in free form, the reduction in tumor volume being determined 40 days following implantation of a xenograft tumor in mice.

41. The method of claim 24, wherein said administering is effective to produce at least about a 9-fold reduction in in vivo tumor volume relative to that provided by administering the chemotherapeutic drug in combination with the biological agent both in free form, the reduction in tumor volume being determined 60 days following implantation of a xenograft tumor in mice.

42. A method of treating a subject for a cancer derived from over-expression of a tyrosine kinase receptor, comprising
administering to the subject (i) a sub-therapeutic amount of an anthracycline antibiotic entrapped in liposomes formed of a vesicle-forming lipid and including a lipid derivatized with a hydrophilic polymer chain to form a liposome-surface coating of hydrophilic polymer chains, and (ii) a dose of a biological agent in free form, said agent having binding activity with tyrosine-kinase receptors on the cancer cells, said dose of biological agent being effective to potentiate the anti-tumor activity of the liposome-entrapped antibiotic,
wherein said administering is effective to produce at least about a 3.8-fold reduction in in vivo tumor volume relative to that provided by administering the chemotherapeutic drug in combination with the biological agent both in free form, the reduction in tumor volume being determined 40 days following implantation of a xenograft tumor in mice.

43. A method of treating a subject having a B-cell-derived lymphoma, comprising
administering to the subject (i) a sub-therapeutic amount of an anthracycline antibiotic entrapped in liposomes formed of a vesicle-forming lipid and including a lipid derivatized with a hydrophilic polymer chain to form a liposome-surface coating of hydrophilic polymer chains, and (ii) a dose of a biological agent in free form, said agent having binding activity to surface epitopes on cells of the B-cell derived lymphoma, said dose of biological agent being effective to potentiate the anti-tumor activity of the liposome-entrapped antibiotic,
wherein said administering is effective to produce at least about a 3.8-fold reduction in in vivo tumor volume relative to that provided by administering the chemotherapeutic drug in combination with the biological agent both in free form, the reduction in tumor volume being determined 40 days following implantation of a xenograft tumor in mice.

44. A method of treating a subject suffering from cancer, comprising
administering to the subject a chemotherapeutic agent entrapped in a liposome; and
administering an anti-angiogenesis biological agent in free form,
wherein said administering is effective to produce at least about a 3.8-fold reduction in in vivo tumor volume relative to that provided by administering the chemotherapeutic drug in combination with the biological agent both in free form, the reduction in tumor volume being determined 40 days following implantation of a xenograft tumor in mice.

45. A method for potentiating the activity of doxorubicin administered in combination with a biological agent to a subject suffering from cancer, comprising;
entrapping the doxorubicin in a liposome, and
administering the liposome-entrapped doxorubicin with the biological agent in free form,
wherein said administering is effective to produce at least about a 2-fold reduction in in vivo tumor volume relative to that provided by administering the chemotherapeutic drug in combination with the biological agent both in free form, the reduction in tumor volume being determined 25 days following implantation of a xenograft tumor in mice.
